(19) United States
(12) Patent Application Publication
(10) Pub. No.: US 2006/0229322 A1
(43) Pub. Date: Oct. 12, 2006

(54) Anhydrous crystalline form of valacyclovir hydrochloride

(76) Inventor: Pau Cid, Barcelona (ES)

Correspondence Address: NIXON & VANDERHYE, PC, 901 NORTH GLEBE ROAD, 11TH FLOOR, ARLINGTON, VA, 22203, US

(21) Appl. No.: 10/537,989

(22) PCT Filed: Dec. 9, 2003
(86) PCT No.: PCT/EP03/13951

§ 371 Date: Dec. 14, 2005

(30) Foreign Application Priority Data
Dec. 9, 2002 (EP) 02258456.9

(51) Int. Cl. A61K 31/522; C07D 473/10

(52) U.S. Cl. 514/263.38; 544/276

(57) ABSTRACT

Valacyclovir hydrochloride in anhydrous crystalline form having substantially the following d-spacing pattern (in angstroms) (see (I)).

The present invention relates to an anhydrous crystalline form of valacyclovir hydrochloride and to its uses and processes for the production thereof.

Valacyclovir is an L-valine ester with 9-[(2-hydroxyethoxy)methyl]guanine. It has the structure shown below

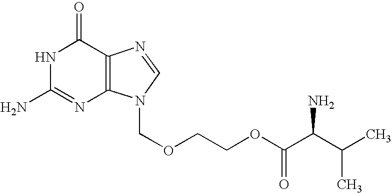

The preparation of valacyclovir is known and is described in, for example, U.S. Pat. No. 4,957,924 and its hydrochloride, which normally occurs as a hydrate, is now widely used as an antiviral agent, especially in the treatment of herpes viruses. Valacyclovir is a prodrug of acyclovir and hence has similar adverse effects, however, valacyclovir is a more bioavailable drug than its counterpart acyclovir and is absorbed more readily in the gastrointestinal tract following oral administration. Whilst valacyclovir hydrochloride is normally administered as a hydrate, it has been found to exist in other forms.

In U.S. Pat. No. 6,107,302 an anhydrous crystalline form of valacyclovir hydrochloride is described which is prepared by contacting valacyclovir hydrochloride with 15 to 40% w/w of a lower alcohol or ketone. The resulting anhydrous crystalline valacyclovir CONFIRMATION COPY hydrochloride is characterised by its X-ray diffraction spectrum having characteristic d-spacing as follows: approximately 24.8, 10.25, 8.14, 7.31, 6.41, 5.85, 5.38, 5.24, 4.89, 4.43, 4.07, 3.71, 3.40, 3.31, 2.92 and 2.78 angstroms.

Hence, the hydrochloride form in U.S. Pat. No. 6,107,302 has distinctive peaks at diffraction angles 2θ=approximately 8.62, 10.86, 12.12, 14.50 and 23.95°.

The present inventors have surprisingly found a new anhydrous crystalline form of valacyclovir hydrochloride which differs from those known in the prior art and which exhibits excellent stability both at room and elevated temperatures.

Hence, viewed from one aspect the invention provides valacyclovir hydrochloride in anhydrous crystalline form having substantially the following d-spacing pattern (in angstroms):

d-spacing

6.76
9.36
11.54
13.98
15.45
15.75
17.12
19.10
21.39
23.02
24.23
26.41
27.46
28.06

Viewed from another aspect the invention provides a pharmaceutical composition comprising a valacyclovir hydrochloride form as hereinbefore described along with one or more pharmaceutical carriers/excipients.

Viewed from another aspect the invention provides a valacyclovir hydrochloride form as hereinbefore described for use in medicine, e.g. as an antiviral agent.

Viewed from a still yet further aspect the invention provides the use of a valacyclovir hydrochloride form as hereinbefore described in the manufacture of a medicament for use as an antiviral agent.

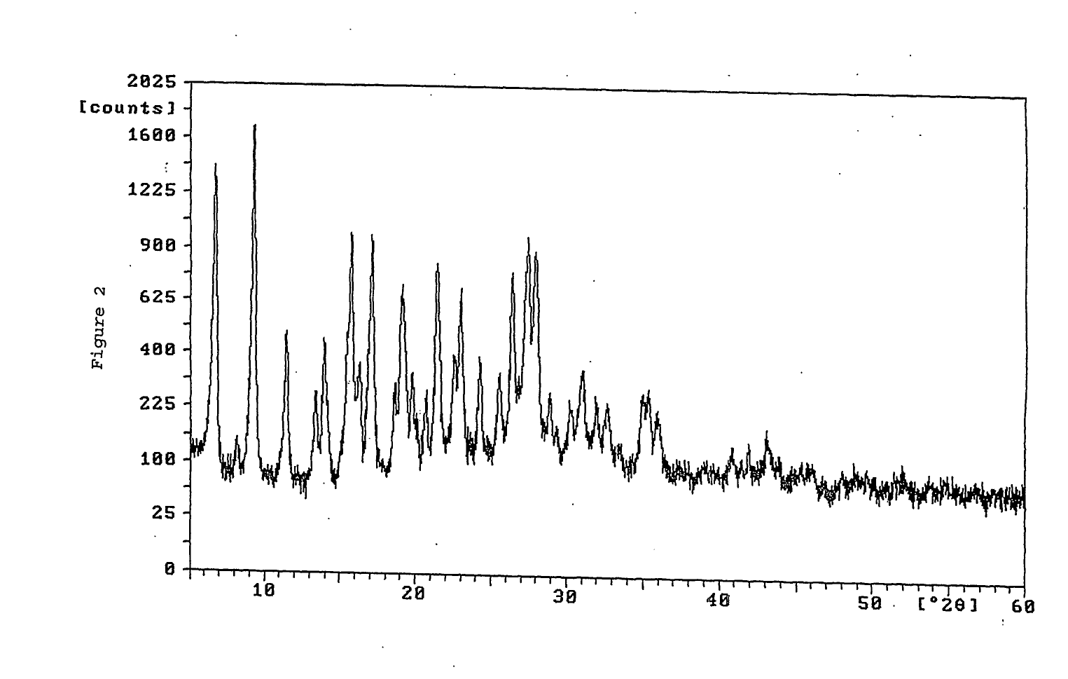

By anhydrous crystalline form according to the invention is meant a crystalline form having substantially the same X-ray powder diffraction pattern as hereinbefore described and shown in FIG. 2. The valacyclovir hydrochloride form should be substantially free of any waters of hydration. The water content of the valacyclovir hydrochloride form of the invention should be below 1.5%, preferably below 1%, for example between 0.5% and 1%, e.g. 0.9% by weight. Water content can be measured using standard Karl-Fischer apparatus (e.g. as described in the 1990 US Pharmacopoeia at pages 1619-1621).

The infrared spectrum of the valacyclovir hydrochloride form of the invention should preferably be substantially as described below:

(the underlined peaks are considered the most characteristic of the anhydrous crystalline form of the invention)

IR (cm⁻¹): 3377.99, 3285.87, 3197.62, 2930.92, 1749.72, 1686.42, 1631.12, 1607.17, 1572.60, 1533.52, 1476.48, 1364.98, 1298.63, 1258.79, 1248.27, 1225.22, 1132.81, 1097.06, 778.37, 759.33.

The person skilled in the art will appreciate that wavenumber measurements may vary slightly on different acquisitions or from machine to machine. Hence, the IR spectrum of the valacyclovir hydrochloride form of the invention should be substantially as described above, i.e. wavenumbers should be within 1% of the given values, preferably within 0.5% of the given values.

Thus, viewed from a further aspect the invention provides valacyclovir hydrochloride in anhydrous crystalline form having substantially the characteristic infrared peaks described above.

The X-ray diffraction pattern of the valacyclovir hydrochloride form may have substantially the following peaks (intensities may vary due to preferred orientation).

| d-spacing | Rel. Int % |
|---|---|
| 6.76 | 79.7 |
| 9.36 | 100.0 |
| 11.54 | 21.1 |
| 13.98 | 23.7 |
| 15.45 | 23.5 |
| 15.75 | 57.4 |
| 17.12 | 59.0 |
| 19.10 | 38.7 |
| 21.39 | 46.4 |
| 23.02 | 39.7 |
| 24.23 | 20.6 |
| 26.41 | 46.4 |
| 27.46 | 55.5 |
| 28.06 | 47.8 |

X-ray diffraction measurements should be taken using a standard Kα1 (Cu λ=1.5406 angstroms) radiation. Full details of the apparatus used may be found in the experimental section.

The person skilled in the art will appreciate that d-spacing measurements may vary slightly on different acquisitions or from machine to machine. Hence, the d-spacing measurements of the valacyclovir hydrochloride form of the invention should be substantially as described above, i.e. d-spacing figures should be within 1% of the given values, preferably within 0.5% of the given values.

The new anhydrous crystalline forms of the invention may be prepared from hydrated valacyclovir hydrochloride. Such a hydrate should be suspended in a lower alcohol, e.g. C₁₋₆-alcohol, preferably C₁₋₄-alcohol, especially ethanol which is substantially pure, i.e. substantially free of any other solvent or water. Preferably therefore, the solvent should be at least 99% lower alcohol, especially 99.8% lower alcohol, e.g. absolute ethanol.

The lower alcohol/hydrochloride mixture should then be heated at elevated temperature e.g. between 50-70° C., preferably 60° C. for at least 12 hours, preferably 15-24 hours, e.g. 20-21 hours. The solvent may then be evaporated under reduced pressure, for example at 60° C. and the crystalline solid thus obtained is valacyclovir hydrochloride as the new anhydrous crystalline form of the invention.

In an alternative embodiment the suspension of lower alcohol and valacyclovir hydrochloride in hydrated form may be added to refluxing lower alcohol, e.g. over 30 minutes. The refluxing alcohol should also be substantially pure as described above. Approximately one third of the solvent is then distilled off and the resulting suspension kept at room temperature, e.g. 20-25° C. for at least 8 hours, e.g. 8-16 hours. The resulting solid may be filtered and washed with substantially pure lower alcohol, e.g. absolute ethanol and dried, e.g. under vacuum at 60° C. An anhydrous crystalline form of valacyclovir hydrochloride of the invention is formed.

Once a seed crystal of valacyclovir hydrochloride in anhydrous crystalline form as claimed herein has been formed it is possible to produce more valacyclovir hydrochloride form of the invention using seeding techniques. For example, valacyclovir hydrochloride dihydrate can be suspended in substantially pure lower alcohol, e.g. isopropanol or isobutanol, and seeded with the anhydrous crystalline form. The suspension should be refluxed for a number of hours and filtered whilst still hot. After drying, the resulting solid is the anhydrous crystalline form of the invention.

Hence, viewed from a still yet further aspect the invention provides a process for the preparation of valacyclovir hydrochloride in anhydrous crystalline form having the X-ray diffraction pattern as hereinbefore described comprising;

1) mixing valacyclovir hydrochloride hydrate with a substantially pure C₁₋₆ lower alcohol solvent and heating the resulting suspension;

2) evaporating the solvent under reduced pressure and isolating the resulting solid.

Alternatively, the invention provides a process for the preparation of valacyclovir hydrochloride in anhydrous crystalline form having the X-ray diffraction pattern as hereinbefore described comprising;

1) mixing valacyclovir hydrochloride hydrate with a substantially pure C₁₆ lower alcohol solvent and adding the resulting suspension to substantially pure refluxing lower alcohol;

2) distilling off the solvent to form a suspension and maintaining the same at room temperature for at least 8 hours; and

3) isolating the resulting solid.

The valacyclovir hydrochloride forms of the invention exhibit remarkable stability and have been shown to be stable at room temperature for over 3 years. Such stability is vital for a pharmaceutical since stocks thereof may be stored for long periods prior to use. Moreover, when heated at 100° C., the new form of valacyclovir hydrochloride exhibits stability for over 550 hours. The valacyclovir hydrochloride form of the invention therefore also shows surprising heat stability, again a property of critical importance to a pharmaceutical. The forms of the invention are therefore suitable for transport in countries where ambient temperatures are exceptionally high.

The valacyclovir hydrochloride form described above may be formulated and employed in medical treatment as is well known in the art. For example, valacyclovir hydrochloride may be employed as an antiviral agent especially in the treatment of diseases caused by various DNA viruses, such as herpes infections, for example, herpes simplex I and II, varicella zoster, cytomegalovirus, Epstein-Barr viruses or human herpes virus-6 as well as diseases caused by hepatitis B. The active compound can also be used for the treatment of papilloma or wart virus infections and may therefore be administered in combination with other therapeutic agents for example with zidovudine, to treat retroviral associated infections in particular HIV infection.

Pharmaceutical preparations may take the form of, inter alia, tablets, capsules, powders, solutions, suspensions or emulsions and they be formulated using standard techniques. While any administration route is possible the preferred route of administration is orally. The new valacyclovir hydrochloride form of the invention may be administered alone or in combination with other forms of valacyclovir hydrochloride or together with any other active ingredients. The amount of valacyclovir hydrochloride administered will vary depending on the patient but will be readily determined by the person skilled in the art. Dosage regimes are known.

The invention will now be described further with reference to the following non-limiting examples and figures.

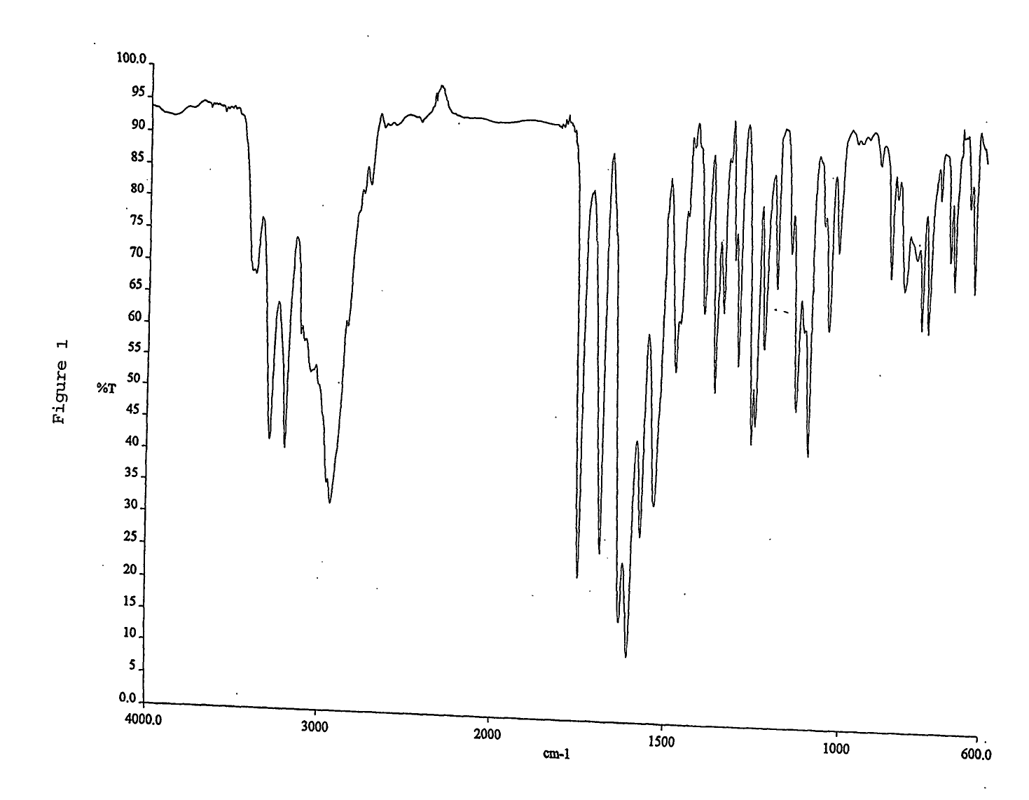

FIG. 1 is the infrared spectrum of the valacyclovir hydrochloride form of the invention produced in Example 1;

FIG. 2 is the X-ray diffraction spectrum of the valacyclovir hydrochloride form of the invention produced in Example 1.

### EXAMPLE 1

3 g of valacyclovir hydrochloride (hydrated form) was suspended in 8 ml of ethanol 99.8% and heated at 60° C. for 20-21 hours. The solvent was evaporated at 60° C. under reduced pressure and the crystalline solid thus obtained was characterised as a new anhydrous crystalline form of valacyclovir hydrochloride.

Nicolet Avatar 320 (FT-IR)

IR (cm⁻¹): 3377.99, 3285.87, 3197.62, 2930.92, 1749.72, 1686.42, 1631.12, 1607.17, 1572.60, 1533.52, 1476.48, 1397.59, 1364.98, 1342.17, 1298.63, 1258.79, 1248.27, 1225.22, 1191.99, 1151.67, 1132.81, 1097.06, 1042.51, 1017.65, 868.28, 829.01, 778.37, 759.33, 729.45, 700.52, 688.32, 631.20.

XRD:

Diffractometer: PW1710 BASED

Tube Anode: Cu

Generator tension [kV]: 40

Generator current [mA]: 40

Wavelength alpha 1: 1.54060

Wavelength alpha 2: 1.54439

Intensity ratio alpha 2/alpha 1: 0.500

Divergence slit: 1.5

Receiving slit: 0.2

Monochromator used: Yes

Start angle [0.52θ]: 5.000

End angle [0.52θ]: 60.000

Step [0.52θ]: 0.2

Maximum intensity: 1536.640

Time per step [s]: 1.000

Peak positions defined by: Minimum of 2nd derivative peak

Minimum peak tip width: 0.00

Maximum peak tip width: 1.00

Peak base width: 2.00

Minimum significance: 0.75

No. of peaks: 47

Data:

| Angle | d-value | d-value | Peak width | Peak int | Back. int | Rel. int | |
|---|---|---|---|---|---|---|---|
| [½2\tilde{0}] | Á1 [] | Á2 [] | [½2\tilde{0}] | [counts] | [counts] | [%] | Signif |
| 6.765 | 13.0556 | 13.0877 | 0.140 | 1225 | 98 | 79.7 | 10.2 |
| 8.160 | 10.8265 | 10.8532 | 0.200 | 61 | 81 | 4.0 | 1.5 |
| 9.360 | 9.4410 | 9.4643 | 0.140 | 1537 | 77 | 100.0 | 12.4 |
| 11.540 | 7.6620 | 7.6808 | 0.140 | 324 | 71 | 21.1 | 4.6 |
| 13.455 | 6.5755 | 6.5917 | 0.080 | 193 | 67 | 12.6 | 0.8 |
| 13.980 | 6.3297 | 6.3453 | 0.140 | 365 | 67 | 23.7 | 4.5 |
| 15.445 | 5.7325 | 5.7466 | 0.120 | 361 | 67 | 23.5 | 1.5 |
| 15.750 | 5.6221 | 5.6360 | 0.180 | 882 | 67 | 57.4 | 12.3 |
| 16.305 | 5.4320 | 5.4453 | 0.100 | 292 | 67 | 19.0 | 1.2 |
| 17.125 | 5.1737 | 5.1864 | 0.180 | 906 | 67 | 59.0 | 12.0 |
| 18.640 | 4.7565 | 4.7682 | 0.120 | 225 | 67 | 14.6 | 1.3 |
| 19.105 | 4.6417 | 4.6531 | 0.120 | 595 | 67 | 38.7 | 2.1 |
| 19.780 | 4.4848 | 4.4959 | 0.100 | 269 | 67 | 17.5 | 1.3 |
| 20.135 | 4.4065 | 4.4174 | 0.120 | 108 | 67 | 7.0 | 0.9 |
| 20.740 | 4.2793 | 4.2899 | 0.060 | 210 | 67 | 13.7 | 1.0 |
| 21.390 | 4.1508 | 4.1610 | 0.140 | 713 | 67 | 46.4 | 4.1 |
| 22.525 | 3.9441 | 3.9538 | 0.100 | 299 | 66 | 19.5 | 0.8 |
| 23.025 | 3.8596 | 3.8691 | 0.060 | 610 | 66 | 39.7 | 2.7 |
| 24.235 | 3.6695 | 3.6786 | 0.120 | 317 | 66 | 20.6 | 2.1 |
| 24.795 | 3.5879 | 3.5967 | 0.100 | 79 | 66 | 5.2 | 0.9 |
| 25.525 | 3.4869 | 3.4955 | 0.200 | 243 | 66 | 15.8 | 3.0 |
| 26.415 | 3.3714 | 3.3797 | 0.140 | 713 | 66 | 46.4 | 4.2 |
| 27.460 | 3.2455 | 3.2534 | 0.100 | 853 | 66 | 55.5 | 1.7 |
| 28.060 | 3.1774 | 3.1852 | 0.200 | 734 | 66 | 47.8 | 8.24 |
| 28.875 | 3.0896 | 3.0972 | 0.200 | 182 | 66 | 11.9 | 2.16 |
| 29.350 | 3.0406 | 3.0481 | 0.160 | 117 | 66 | 7.6 | 0.90 |
| 30.195 | 2.9574 | 2.9647 | 0.160 | 166 | 64 | 10.8 | 0.99 |
| 31.045 | 2.8784 | 2.8855 | 0.100 | 282 | 64 | 18.4 | 1.16 |
| 31.950 | 2.7989 | 2.8058 | 0.200 | 164 | 64 | 10.7 | 1.77 |
| 32.620 | 2.7429 | 2.7496 | 0.100 | 185 | 64 | 12.0 | 0.79 |
| 33.470 | 2.6752 | 2.6817 | 0.320 | 72 | 64 | 4.7 | 1.19 |
| 34.915 | 2.5677 | 2.5740 | 0.200 | 190 | 64 | 12.4 | 2.19 |
| 35.340 | 2.5378 | 2.5440 | 0.160 | 204 | 64 | 13.3 | 1.43 |
| 35.945 | 2.4964 | 2.5026 | 0.240 | 151 | 64 | 9.8 | 1.48 |
| 38.940 | 2.3110 | 2.3167 | 0.480 | 37 | 62 | 2.4 | 1.18 |
| 40.805 | 2.2096 | 2.2150 | 0.120 | 81 | 62 | 5.3 | 0.81 |
| 41.440 | 2.1772 | 2.1826 | 0.160 | 56 | 62 | 3.7 | 0.84 |
| 41.905 | 2.1541 | 2.1594 | 0.160 | 81 | 62 | 5.3 | 1.11 |
| 43.085 | 2.0978 | 2.1030 | 0.200 | 102 | 62 | 6.6 | 1.46 |
| 43.920 | 2.0599 | 2.0649 | 0.240 | 44 | 62 | 2.8 | 0.77 |
| 46.115 | 1.9668 | 1.9716 | 0.240 | 41 | 62 | 2.7 | 1.09 |
| 48.055 | 1.8918 | 1.8965 | 0.240 | 29 | 61 | 1.9 | 0.87 |
| 48.895 | 1.8613 | 1.8658 | 0.400 | 28 | 61 | 1.8 | 0.78 |
| 51.665 | 1.7678 | 1.7721 | 0.640 | 21 | 61 | 1.4 | 0.93 |
| 53.715 | 1.7051 | 1.7093 | 0.240 | 22 | 61 | 1.4 | 1.02 |
| 54.800 | 1.6738 | 1.6780 | 0.320 | 21 | 61 | 1.4 | 0.86 |
| 56.740 | 1.6211 | 1.6251 | 0.480 | 12 | 59 | 0.8 | 1.25 |

### EXAMPLE 2

Valacyclovir hydrochloride (hydrated form) was suspended in ethanol 99.8%. The suspension was added over about 30 minutes to refluxing ethanol 99.8%. The solvent was distilled off and the suspension kept at 20-25° C. for 12 hours. The solid was filtered, washed twice with absolute ethanol and dried under vacuum at 60° C. The crystalline solid thus obtained was characterised as a new anhydrous crystalline form of valacyclovir hydrochloride.

### EXAMPLE 3

10 g of valacyclovir hydrochloride dihydrate (KF 6%) was suspended in 100 ml of isopropanol 99% and seeded with 50 mg of anhydrous crystalline valacyclovir hydrochloride. The suspension was refluxed for 5 hours and filtered hot. The solid was dried at 110° C. for 10 hours (yield=7.52 g).

### EXAMPLE 4

10 g of valacyclovir hydrochloride dihydrate (KF 6%) was suspended in 100 ml of isobutanol 99% and seeded with 50 mg of anhydrous crystalline valacyclovir hydrochloride. The suspension was refluxed for 5 hours and filtered hot. The solid was dried at 110° C. for 10 hours (yield=8.24 g).

1. Valacyclovir hydrochloride in anhydrous crystalline form having substantially the following d-spacing pattern (in angstroms):
d-spacing
6.76
9.36
11.54
13.98
15.45
15.75
17.12
19.10
21.39
23.02
24.23
26.41
27.46
28.06

2. Valacyclovir hydrochloride in anhydrous crystalline form as claimed in claim 1 having substantially the X-ray diffraction pattern of FIG. 2.

3. Valacyclovir hydrochloride in anhydrous crystalline form having substantially the characteristic infrared peaks
IR (cm⁻¹): 1686.42, 1572.60, 1533.52.

4. Valacyclovir hydrochloride in anhydrous crystalline form as claimed in claim 3 having substantially the characteristic infrared peaks
IR (cm⁻¹): 3377.99, 3285.87, 3197.62, 2930.92, 1749.72, 1686.42, 1631.12, 1607.17, 1572.60, 1533.52, 1476.48, 1364.98, 1298.63, 1258.79, 1248.27, 1225.22, 1132.81, 1097.06, 778.37, 759.33.

5. Valacyclovir hydrochloride in anhydrous crystalline form as claimed in claim 3 having substantially the infra-red absorption spectrum of FIG. 1.

6. A pharmaceutical composition comprising a valacyclovir hydrochloride form as claimed in claim 1 along with one or more pharmaceutical carriers/excipients.

7. Valacyclovir hydrochloride in anhydrous crystalline form as claimed in claim 1 for use in medicine.

8. Use of valacyclovir hydrochloride in anhydrous crystalline form as claimed in claim 1 in the manufacture of a medicament for use as an antiviral agent.

9. A process for the preparation of valacyclovir hydrochloride in anhydrous crystalline form as claimed in claim 1 comprising;
1) mixing valacyclovir hydrochloride hydrate with a substantially pure C₁₋₆ lower alcohol solvent and heating the resulting suspension;
2) evaporating the solvent under reduced pressure and isolating the resulting solid.

10. The process of claim 9 wherein said solvent is ethanol.

11. The process of claim 9 wherein the suspension is heated at between 50 to 70° C. for at least 12 hours.

12. The process of claim 11 wherein the suspension is heated at 60° C. for 20-21 hours.

13. A process for the preparation of valacyclovir hydrochloride in anhydrous crystalline form as claimed in claim 1 comprising;
1) mixing valacyclovir hydrochloride hydrate with a substantially pure C₁₋₆ lower alcohol solvent and adding the resulting suspension to substantially pure refluxing lower alcohol;
2) distilling off the solvent to form a suspension and maintaining the same at room temperature for at least 8 hours; and
3) isolating the resulting solid.

14. The process of claim 13 wherein the solvent and refluxing lower alcohol are ethanol.

15. The process of claim 13 wherein approximately one third of the solvent is distilled off to form said suspension.
